(19) United States
(12) Patent Application Publication
(10) Pub. No.: US 2015/0017128 A1
(43) Pub. Date: Jan. 15, 2015

(54) STRAINS OF LACTIC ACID BACTERIA AND/OR BIFIDOBACTERIA INHIBITING/REDUCING THE GROWTH OF DIFFERENT BIOTYPES OF E. COLI AND DIFFERENT BIOTYPES OF CLOSTRIDIA

(71) Applicant: Giovanni Mogna, Novara (IT)

(72) Inventor: Giovanni Mogna, Novara (IT)

(21) Appl. No.: 14/344,047

(22) PCT Filed: Sep. 10, 2012
(86) PCT No.: PCT/IB2012/001745

§ 371 Date: Jul. 28, 2014

(30) Foreign Application Priority Data
Sep. 9, 2011 (IT) RM2011A000475

(51) Int. Cl. A61K 35/74; A61K 38/47; A23L 1/30; A61K 31/198

(52) U.S. Cl. CPC A61K 35/747; A61K 35/745; A61K 31/198; A61K 38/47; A23L 1/3014; A23Y 2220/65; A23Y 2220/73; A23V 2002/00
USPC 424/93.3; 424/93.45; 424/93.4; 424/498

(57) ABSTRACT

The present disclosure refers to strains of lactic bacteria and/or bifidobacteria having activity of inhibiting/reducing the growth of different biotypes of E. coli, including E. coli 0157:7 and different biotypes of clostridia, including Clostridium difficile, Listeria monocytogenes, Enterococcus sp. and Klebsiella sp. Furthermore, the present invention refers to a pharmaceutical or dietary composition or a supplement or a medical device with at least one of the said strains of bacteria, optionally in combination with acetylcysteine and/or microencapsulated gastroprotected lysozyme and/or acetylcysteine with microencapsulated gastro-protected lysozyme.

The present invention refers to strains of lactic bacteria and bifidobacteria, having actions of inhibiting/reducing the growth of different biotypes of E. coli (gram-negative), including E. coli O157:H7 and O104:H4, and of different bacterial species belonging to the family of the Clostridiaceae (anaerobic, Gram positive rods) responsible for putrefaction and serious intestinal infections (Clostridium difficile).

Furthermore, the present invention refers to strains of lactic bacteria having specific antimicrobial activity against Listeria monocytogenes (gram-positive), Enterococcus sp. and Klebsiella sp. (gram-negative). Furthermore, the present invention refers to a pharmaceutical or dietary composition or a supplement or a medical device comprising at least one of said strains of lactic bacteria and/or bifidobacteria, optionally in combination with acetylcysteine and/or lysozyme.

It is known that Escherichia coli—abbreviated to E. coli—is the best-known species of the genus Escherichia: within it, at least 171 serotypes are distinguished, characterised by different combinations of the antigens O, H, K, F. It is one of the principal species of bacteria which live in the lower part of the intestine of warm-blooded animals (birds and mammals, including man), and which are necessary for the correct digestion of food. Its presence in bodies of water indicates the presence of faecalisation conditions (it is the principal indicator of faecal contamination, together with the enterococci). The genus Escherichia, together with other genera such as for example Enterobacter, Klebsiella, Citrobacter and Serratia, are grouped together under the name coliforms. Technically the “coliform group” comprises non-sporogenous aerobic and anaerobic bacteria.

Even though it is a common inhabitant of the intestine and has a fundamental role in the digestive process, there are situations in which E. coli can cause illnesses in man and animals. Some strains of E. coli are the causative agent of intestinal and extra-intestinal illnesses such as infections of the urinary tract, meningitis, peritonitis, septicaemia and pulmonitis. Some strains of E. coli are toxigenic, i.e. they produce toxins which can be the cause of diarrhoea among other things. Dysentery caused by E. coli is a common alimentary toxinfection, since it is contracted principally from contaminated foodstuffs. Contamination can occur from inadequately-cooked infected meat, from unpasteurised milk and cheeses made from it, and from other foodstuffs contaminated by faeces. E. coli produces four types of toxins which are distinguished, by their different sensitivity to thermal treatment, into thermolabile and thermostable, and by their toxigenic action (Shiga toxins and haemolytic toxins, HlyA).

The thermolabile toxin, named LT, is very similar in structure and functions to the cholera toxin. It contains an ‘A’ subunit and five ‘B’ subunits in a holotoxin. The B subunits contribute to adherence and to the toxin's entry into the intestinal cells of the host, where the A subunit stimulates the cells to release water, causing diarrhoea.

A “strain” of E. coli is a group with particular characteristics which are able to make it recognisable by other strains of E. coli, similarly to the way animals of different breeds can be distinguished. Different strains of E. coli live in different animal species, so that it is possible to establish whether faecal material in water comes, for example, from human beings or birds.

New strains of E. coli arise continually from the natural biological process of mutation, and some of these strains have characteristics which may be harmful to a host animal. Although in the majority of adult humans a pathogenic strain would probably not cause anything but diarrhoea, and could not produce any symptoms, in small children or sick people or those debilitated by recent illnesses or undergoing particular treatments, a new strain could cause serious illnesses and even death. An example of a particularly virulent strain of E. coli is E. coli O157:H7. The strains of enterohaemorrhagic E. coli are the principal ones responsible for illness in industrialised countries. The serotype principally responsible for these illnesses is E. coli O157:H7.

E. coli O157: H7 or enterohaemorrhagic Escherichia coli (EHEC) is an enterohaemorrhagic strain of the bacterium Escherichia coli which is a cause of diseases transmitted by food. Infection often leads to haemorrhagic diarrhoea and, occasionally, to renal failure, haemolytic-uraemic syndrome (HUS), especially in young people, children and old people. Transmission occurs by the faecal-oral route, and the majority of cases of illness are associated with dietary consumption of raw or under-cooked foods, contaminated by the soil or contaminated water, or consumption of vegetables polluted by such water. In ruminants it lives as a commensal in the intestine, but does not give rise to pathology because of the scarcity of receptors for the toxin produced by the bacterium. With reference to the genus Clostridium, it is known that many foods contain these bacteria which are responsible for the degradation processes of the foods themselves, and that if they are ingested with the diet and reach the intestine, they are able to modify the composition of the microflora and initiate putrefaction.

The foods generally responsible for these alimentary toxinfections are fresh meats contaminated in particular by Clostridium perfringens, C. sporogenes, C. bifermentans, etc.), which can hydrolyse sugars forming malodorous substances such as indole, ammonia and mercaptans.

Fish, on the other hand, can be attacked by another species of Clostridium, C. botulinum which can develop and produce a toxin even at low temperatures (4° C.). Among the Clostridia most dangerous to man is Clostridium difficile, which can produce enterotoxins responsible for cases of haemorrhagic diarrhoea, pseudomembranous colitis with serious complications such as perforation, septicaemia and death. The bacterium forms spores resistant to heat and is transmitted from person to person via the faecal-oral route.

Generally the pathogenesis caused by C. difficile is associated with the use of antibiotics which cause marked imbalance in the intestinal microflora, causing the prevalence of putrefacient species, to the detriment of those with fermentative action.

C. difficile is ubiquitous, in particular in hospital environments, and can colonise the intestine of a small percentage of healthy individuals (about 5%). The use of antibiotics is associated with its excessive development and with its serious pathological manifestations due to its resistance to the majority of antibiotics. The spores ingested, once they have overcome the gastric barrier, settle in the colon where they germinate and evolve into the vegetative form, which by binary multiplication generates a numerous population responsible for the multiple pathological forms which are particularly serious in the elderly.

It is estimated that currently this organism infects more than 30% of patients treated in hospitals in the United States. In addition, Listeria monocytogenes is a ubiquitous pathogen responsible in man for listeriosis, a potentially fatal infection of alimentary origin.

Probiotic bacteria (lactobacilli and bifidobacteria) are micro-organisms which benefit the health of the host, and for this reason are commonly consumed by an ever-increasing number of people who choose food products, fermented milk products, yogurt and supplements containing probiotic bacteria.

There remains a need, therefore, to have available probiotic bacteria capable of colonising the intestine and bringing beneficial effects to the host. In particular, there remains a need to have available probiotic bacteria capable of prevailing over pathogenic species, with consequent health benefits. Even more particularly, there remains a need to have available probiotic bacteria capable of preventing and/or treating infections and/or pathologies connected with pathogenic bacteria (gram-positive and gram-negative).

Following intensive research activity, the Applicant has surprisingly found that selected bacteria are capable of giving an appropriate response to the above-mentioned needs.

The Applicant has, furthermore, surprisingly found that by using N-acetylcysteine, which has the characteristic of dissolving the bacterial biofilms normally produced in self-defence by the pathogenic germs, the bacteriocin action of the selected probiotic strains is much more effective.

It has furthermore surprisingly found that by using a particular technique of microencapsulation in a lipid matrix, preferably of a vegetable nature, which confers gastroprotection on the lysozyme, the latter is enabled to pass undamaged through the gastroduodenal tract, to be activated at colon level and to express its lytic action against the spores of all species of clostridia. As a consequence of this hydrolytic action on the spores of the genus Clostridium and C. difficile in particular, at the moment of their germination, the formation of vegetative cells responsible for the increase in the pathogen population and the formation of toxins does not occur.

The combined action of probiotic strains with fermentative activity and barrier effect against the putrefacient species, including the clostridia, together with the direct action of the lysozyme which in fact prevents the germination of the clostridia spores, constitutes an extraordinary and innovative strategy against this dangerous group of pathogens.

The total combination (probiotic bacteria of the present invention producing bacteriocins, and/or N-acetylcysteine which dissolves the bacterial biofilm, and/or lysozyme, preferably in microencapsulated form in a lipid matrix, which blocks the germination of the spores) constitutes an innovative tool capable of combating a large number of pathogenic strains, against which up to now there was no effective strategy of opposition.

A subject of the present invention is formed by a selection of strains of lactic bacteria and/or bifidobacteria which are capable of producing bacteriocins and/or metabolites and/or oxygenated water for use in the preventive and/or curative treatment of infections and/or pathologies connected with: the various pathogens, including E. coli and in particular E. coli O157:H7 and O104:H4; and/or putrefacient germs belonging to the family of the Clostridiaceae, Clostridium difficile; and/or pathogens Listeria monocytogenes, Enterococcus sp., Klebsiells sp., said bacteria being also, optionaly, used in combination with N-acetylcysteine and/or lysozyme. The lysozyme can be microencapsulated and gastroprotected in a lipid matrix. In a preferred embodiment, the lipid matrix is of vegetable origin having a melting point comprised from 30° C. to 80° C., preferably from 40° C. to 70° C., even more preferably from 50° C. to 60°.

Another subject of the present invention is formed by a pharmaceutical or dietary composition or a supplement or a medical device comprising at least one of said strains of bacteria for use in the preventive and/or curative treatment of infections and/or pathologies connected with E. coli pathogens, including E. coli O157:H7; and/or putrefacient germs belonging to the family of the Clostridiaceae, including Clostridium difficile; and/or pathogens Listeria monocytogenes, Enterococcus sp., Klebsiells sp.

Another subject of the present invention is formed by a pharmaceutical or dietary composition or a supplement or a medical device comprising at least one of the said strains of bacteria in combination with N-acetylcysteine.

Another subject of the present invention is formed by a pharmaceutical or dietary composition or a supplement or a medical device comprising at least one of the said strains of bacteria in combination with lysozyme. In a preferred embodiment, the lysozyme is microencapsulated.

Another subject of the present invention is formed by a pharmaceutical or dietary composition or a supplement or a medical device comprising at least one of the said strains of bacteria in combination with N-acetylcysteine and with lysozyme. In a preferred embodiment, the lysozyme is microencapsulated with a lipid matrix. In a preferred embodiment, the lipid matrix is of vegetable origin having a melting point comprised from 30° C. to 80° C., preferably from 40° C. to 70° C., even more preferably from 50° C. to 60°.

Other preferred embodiments of the present invention are described in the continuation of the description and will be claimed in the attached dependent claims.

### LIST OF TABLES AND DRAWINGS

Table 1 shows a list of 14 strains of lactobacilli, 4 strains of bifidobacteria and 2 strains of streptococci which have been tested against 4 biotypes of E coli.

Tables 2-5 list the strains which produce bacteriocins and/or metabolites and/or oxygenated water and which are able to combat, inhibit or reduce the growth of pathogenic bacteria belonging to the species E. coli, including E. coli O157:H7.

Tables 6 and 7 refer to the strains of the genus Lactobacillus.

FIG. 1 shows the technique used for testing the strains which are the subject of the present invention.

FIG. 2 shows the inhibition halos formed by the strains of Table 4, against E. coli (ATCC 8739 ATCC 10536 ATCC 35218 ATCC 25922) and serotype O157:H7 (CQ 9485).

FIG. 3 refers to the data shown in Tables 6 and 7.

FIG. 4 refers to the in vitro inhibitory activity of the strains of probiotic bacteria against E. coli.

FIGS. 5 and 6 refer to the in vitro inhibitory activity of the strains of probiotic bacteria against other enteropathogenic bacteria.

The Applicant has conducted intense research and screening activity starting from a very extensive group of strains of bacteria. At the end of this activity, the Applicant found and selected a group of strains of bacteria belonging to a species or to several species selected from the group comprising or, alternatively, consisting of Lactobacillus delbrueckii, Lactobacillus delbrueckii subsp. delbrueckii, Lactobacillus plantarum, Lactobacillus rhamnosus, Lactobacillus pentosus and Bifidobacterium breve. The selected strains are capable of producing bacteriocins and/or metabolites and/or oxygenated water, these being substances which are capable of effectively combating, inhibiting or reducing pathogenic bacteria. These strains have a valid application and use in the preventive and/or curative treatment of infections and/or pathologies connected with gram-negative and/or gram-positive pathogenic bacteria.

The pathogenic bacteria are selected from the group comprising the coliforms and clostridia. The coliforms are a group of bacteria belonging to the family of Enterobacteriaceae. In particular, the coliforms are rod-shaped, gram-negative, asporogenous, aerobic and anaerobic facultative bacteria, which ferment lactose, with the production of gas and acids, at 35-37° C. in 48 hours and possess the enzyme beta-galactosidase. They are ubiquitous organisms; some are present in faecal material, and are therefore used as indicators of pollution both of water and of food, others are of aquatic or telluric origin. The group comprises more than fifty genera, among them Citrobacter, Enterobacter, preferably Enterobacter cloacae, Escherichia, preferably E. coli, Hafnia, Klebsiella, preferably Klebsiella pneumoniae, Serratia and Yersinia. Other pathogens always of interest in the context of the present invention belong to the family of the Clostridiaceae, with particular reference to C. difficile. Antagonistic action is exerted also against species selected from the group comprising Salmonella enteriditis, Campylobacter jejuni, Helicobacter pylori, Listeria monocytogenes, Enterococcus sp. and Klebsiella sp.

A subject of the present invention is formed by a pharmaceutical or dietary composition or a supplement or a medical device comprising at least one bacterial strain belonging to a species or several species selected from the group comprising or, alternatively, consisting of: Lactobacillus delbrueckii, Lactobacillus delbrueckii subsp. delbrueckii, Lactobacillus plantarum, Lactobacillus rhamnosus, Lactobacillus pentosus, Lactobacillus reuteri and Bifidobacterium breve, capable of producing bacteriocins and/or metabolites and/or oxygenated water for use in the preventive and/or curative treatment of infections and/or pathologies connected with pathogens such as E. coli, C. difficile, Listeria monocytogenes, Enterococcus sp. and Klebsiella sp.

In one embodiment, the pharmaceutical or dietary composition or supplement or the medical device comprises at least one bacterial strain belonging to a species or several species selected from the group comprising or, alternatively, consisting of: Lactobacillus plantarum, Lactobacillus rhamnosus and Lactobacillus pentosus, capable of producing bacteriocins and/or metabolites and/or oxygenated water for use in the preventive and/or curative treatment of infections and/or pathologies connected with pathogens such as E. coli, C. difficile, Listeria monocytogenes, Enterococcus sp. and Klebsiella sp.

The strain and/or the various possible combinations of the above-mentioned strains (see also Tables 1-5 below) selected for said purpose may be used alone or in combination with N-acetylcysteine; or in combination with microencapsulated lysozyme; or in combination with N-acetylcysteine and microencapsulated lysozyme.

In one embodiment, the pathogen E. coli is selected from among E. coli O157:H7 and E. coli O104:H4.

Table 1 shows a list of 14 strains of lactobacilli, 4 strains of bifidobacteria and 2 strains of streptococci which have been tested against 4 biotypes of E coli. The values are expressed as inhibition halo (mm).

TABLE 1

| | | | Antimicrobial activity against E. coli | | | |
|---|---|---|---|---|---|---|
| | | | ATCC | ATCC | ATCC | ATCC |
| Species | Strain | Filing No. | 8739 | 10536 | 35218 | 25922 |
| B. animalis subsp. | BS01 | LMG P-21384 | + | + | − | − |
| lactis | | | | | | |
| B. breve | BR03 | DSM 16604 | ++ | ++ | + | + |
| B. breve | B632 | DSM 24706 | ++ | ++ | + | + |
| B. longum | BL05 | DSM 23234 | − | − | − | − |
| L. paracasei | LPC00 | LMG P-21380 | − | − | − | − |
| L. paracasei | LPC08 | DSM 21718 | − | − | − | − |
| L. rhamnosus | LR04 | DSM 16605 | ++ | ++ | ++ | +++ |
| L. rhamnosus | LGG | ATCC 53103 | − | + | − | − |
| L. rhamnosus | LR06 | DSM 21981 | ++ | ++ | + | + |
| L. fermentum | LF10 | DSM 19187 | − | − | − | − |
| L. plantarum | LP01 | LMG P-21021 | ++ | ++ | ++ | + |
| L. plantarum | LP02 | LMG P-21020 | + | ++ | + | + |
| L. plantarum | LP03 | LMG P-21022 | ++ | +++ | − | + |
| L. plantarum | LP04 | LMG P-21023 | ++ | +++ | − | + |
| L. pentosus | LPS01 | DSM 21980 | ++ | ++ | +++ | +++ |
| L. delbr. subsp. | LDD01 | DSM 22106 | ++ | +++ | ++ | +++ |
| delbrueckii | | | | | | |
| L. reuteri | LRE01 | DSM 23877 | + | + | − | − |
| L. reuteri | LRE03 | DSM 23879 | + | + | − | − |
| S. thermophilus | YO2 | DSM 16590 | − | − | − | − |
| S. thermophilus | YO3 | DSM 16591 | − | − | − | − |

The bacteria tested in Table 1, taken singly or in combination with each other, have a valid application in the context of the present invention.

In one embodiment, the strains of bacteria are selected from the group comprising or, alternatively, consisting of those listed in Table 2.

TABLE 2

| Species | Strain | Filing No. |
|---|---|---|
| B. breve | BR03 | DSM 16604 |
| B. breve | B632 | DSM 24706 |
| L. rhamnosus | LR04 | DSM 16605 |
| L. rhamnosus | LR06 | DSM 21981 |
| L. plantarum | LP01 | LMG P-21021 |
| L. plantarum | LP02 | LMG P-21020 |
| L. plantarum | LP03 | LMG P-21022 |
| L. plantarum | LP04 | LMG P-21023 |
| L. plantarum | LP04 | LMG P-21023 |
| L. pentosus | LPS01 | DSM 21980 |
| L. delbr. subsp. Delbrueckii | LDD01 | DSM 22106 |
| L. reuteri | LRE01 | DSM 23877 |
| L. reuteri | LRE03 | DSM 23879 |
| L. reuteri | LRE03 | DSM 23879 |
| L. reuteri | LRE03 | DSM 23879 |

The strains of Table 2 are capable of producing bacteriocins and/or metabolites and/or oxygenated water which are able to combat, inhibit or reduce the growth of pathogenic bacteria belonging to the species E. coli, including E. coli O157:H7. The strains of Table 2 have been individually tested for the purpose of identifying their capacity for antagonising (inhibiting the growth or reducing the number of one or more harmful or pathogenic microbial species/genera), as stated in the four columns on the right in Table 1.

All the strains described and/or claimed in the present patent application have been deposited in accordance with the Treaty of Budapest and are put at the disposal of the public on request to the competent Depositing Authority.

In another embodiment, the strains of bacteria are selected from the group comprising or, alternatively, consisting of those listed in Table 3.

TABLE 3

| Species | Strain | Filing No. |
|---|---|---|
| B. breve | BR03 | DSM 16604 |
| B. breve | B632 | DSM 24706 |
| L. rhamnosus | LR04 | DSM 16605 |
| L. rhamnosus | LR06 | DSM 21981 |
| L. plantarum | LP01 | LMG P-21021 |
| L. plantarum | LP02 | LMG P-21020 |
| L. plantarum | LP03 | LMG P-21022 |
| L. plantarum | LP04 | LMG P-21023 |
| L. plantarum | LP04 | LMG P-21023 |
| L. pentosus | LPS01 | DSM 21980 |
| L. delbr. subsp. Delbrueckii | LDD01 | DSM 22106 |

In a further embodiment, the strains of bacteria are selected from the group comprising or, alternatively, consisting of those listed in Table 4.

TABLE 4

| Species | Strain | Filing No. |
|---|---|---|
| B. breve | BR03 | DSM 16604 |
| B. breve | B632 | DSM 24706 |
| L. rhamnosus | LR04 | DSM 16605 |
| L. rhamnosus | LR06 | DSM 21981 |
| L. plantarum | LP01 | LMG P-21021 |
| L. plantarum | LP02 | LMG P-21020 |
| L. pentosus | LPS01 | DSM 21980 |
| L. delbr. subsp. Delbrueckii | LDD01 | DSM 22106 |

The composition of the present invention has further valid application also for use in the preventive and/or curative treatment of infections and/or pathologies connected with the pathogens E. coli, H. pylori and Clostridium difficile.

In one embodiment, the composition of the present invention comprises or, alternatively, consists of from one to eight strains, selected from the group comprising or, alternatively, consisting of those listed in Table 4.

In another preferred embodiment, the composition of the present invention comprises or, alternatively, consists of from one to six strains, preferably of from two to five strains, even more preferably of three or four strains, selected from the group comprising or, alternatively, consisting of the strains listed in Table 4.

In a preferred embodiment, the composition comprises or, alternatively, consists of the six strains, as listed in Table 5; or of the 4 lactobacilli of Table 5.

TABLE 5

| Species | Strain | Filing No. |
|---|---|---|
| B. breve | BR03 | DSM 16604 |
| B. breve | B632 | DSM 24706 |
| L. rhamnosus | LR06 | DSM 21981 |
| L. plantarum | LP01 | LMG P-21021 |
| L. pentosus | LPS01 | DSM 21980 |
| L. delbr. subsp. Delbrueckii | LDD01 | DSM 22106 |

The Applicant has found that the strains of bacteria selected from the group comprising or, alternatively, consisting of L. pentosus LPS01 DSM 21980, L. plantarum LP01 LMG P-21021, L. plantarum LP02 LMG P-21020 and L. rhamnosus LR04 DSM 16605 are able to exert an inhibitory activity against the 4 strains of E. coli; in particular the strains L. rhamnosus LR04 DSM 16605 and L. pentosus LPS01 DSM 21980 show greater activity also against the serotype O157:H7.

Furthermore, the Applicant has found that the strain L. plantarum LP01 LMG P-21021 showed inhibitory capacity against all the enteropathogenic bacteria, in particular against Listeria monocytogenes.

A subject of the present invention is formed by a pharmaceutical or dietary composition or a supplement or a medical device comprising at least one bacterial strain belonging to a species or several species selected from the group comprising or, alternatively, consisting of: Lactobacillus plantarum, Lactobacillus rhamnosus and Lactobacillus pentosus which is capable of producing bacteriocins and/or metabolites and/or oxygenated water for use in the preventive and/or curative treatment of infections and/or pathologies connected with pathogens such as E. coli, including serotype O157:H7, and Listeria monocytogenes.

In one embodiment, said pharmaceutical or dietary composition or supplement or medical device comprises at least one bacterial strain selected from the group comprising or, alternatively, consisting of L. pentosus LPS01 DSM 21980, L. plantarum LP01 LMG P-21021, L. plantarum LP02 LMG P-21020 and L. rhamnosus LR04 DSM 16605; advantageously it comprises or consists of the strains L. rhamnosus LR04 DSM 16605 and L. pentosus LPS01 DSM 21980; or it comprises or consists of the strain L. plantarum LP01 LMG P-21021.

In the composition of the present invention, the mixture of strains of bacteria is present in a quantity comprised between 0.5% and 20% by weight, compared with the total weight of the composition, preferably between 2.5% and 10%. In a preferred embodiment, the composition can furthermore comprise at least one prebiotic fibre and/or carbohydrates with bifidogenic action.

The prebiotic fibre which has application in the composition of the present invention is a fibre which must be used by the strains of bacteria present in the composition, but not by the pathogens which it is intended to antagonise.

Furthermore, the composition can also comprise other active ingredients and/or components such as vitamins, minerals, bioactive peptides, substances with anti-oxidising, hypocholesterolaemic, hypoglycaemic and anti-inflammatory action and anti-sweetening agents in a quantity generally comprised between 0.001% and 20% by weight, preferably between 0.01% and 5% by weight, always depending on the type of active component and its recommended daily dose if any, compared with the total weight of the composition.

The dietary composition which is the subject of the present invention, for example a symbiotic composition, or a supplement or a pharmaceutical composition or a medical device, is prepared according to the techniques and the equipment known to experts in the field.

In a preferred embodiment, the composition contains bacteria in a concentration comprised between 1×10⁶ and 1×10¹¹ CFU/g of mixture, preferably between 1×10⁸ and 1×10¹⁰ CFU/g of mixture.

In a preferred embodiment, the composition contains bacteria in a concentration comprised between 1×10⁶ and 1×10¹¹ CFU/dose, preferably between 1×10⁸ and 1×10¹⁰ CFU/dose.

The dose can be comprised between 0.2 and 10 g, for example it is of 0.25 g, 1 g, 3 g, 5 g or 7 g.

The probiotic bacteria used in the present invention can be in solid form, in particular in the form of powder, dehydrated powder or lyophilized.

All the compositions of the present invention are prepared according to techniques known to experts in the field, and by the use of known equipment.

In a preferred embodiment, the composition which is the subject of the present invention comprises furthermore acetylcysteine (or N-acetylcysteine) which is an N-acetylated derivative of the amino acid cysteine, and lysozyme, preferably microencapsulated and gastroprotected in a lipid matrix, as described above.

The Applicant has found that the use of acetylcysteine in association with one or two of the strains of bacteria, described in Tables 1-5, or in the various preferred embodiments mentioned above, is capable of dissolving the bacterial biofilm produced by the pathogenic bacteria themselves and which is used by said pathogens as protection. In practice it has been seen that the pathogenic bacteria are capable of forming a protective coating (biofilm) around the cells. The biofilm makes the cells of the pathogens more difficult to attack and better protected. Acetylcysteine is capable of penetrating the biofilm of the cells and dissolving it, facilitating the attack on the pathogenic cells by means of the bacteriocins and/or the metabolites and/or the oxygenated water produced by the strains of bacteria which are the subject of the present invention.

The quantity of acetylcysteine present in the composition to be administered, which is the subject of the present invention, is comprised between 10 and 1,000 mg/day, preferably it is comprised between 200 and 600 mg/day. Acetylcysteine, preferably in solid form, is mixed with the probiotic bacteria, preferably in solid or lyophilised form, using techniques and equipment known to experts in the field.

The quantity of lysozyme, preferably microencapsulated, present in the composition to be administered, which is the subject of the present invention, is comprised between 10 and 2.000 mg/day, preferably it is comprised between 400 and 1.000 mg/day. The lysozyme (preferably microencapsulated) is preferably in solid form and is mixed with the probiotic bacteria, preferably in solid or lyophilised form, using techniques and equipment known to experts in the field.

The quantities of acetylcysteine and microencapsulated lysozyme present in the composition to be administered, which is the subject of the present invention, are respectively comprised between 10 and 1,000 mg/day, preferably between 200 and 600 mg/day, while the microencapsulated lysozyme is comprised between 10 and 2,000 mg/day, preferably between 400 and 1,000 mg/day; they are mixed with the probiotic bacteria in solid form, preferably in solid or lyophilised form, using techniques and equipment known to experts in the field. The quantity of N-acetylcysteine present in the composition which is the subject of the present invention is comprised between 10 and 1,000 mg/day, preferably between 50 and 200 mg/day, even more preferably between 60 and 150 mg/day. N-acetylcysteine is available on the market in a pharmaceutically acceptable form, preferably in solid form, and is mixed with the probiotic bacteria, preferably in solid or lyophilised form, using techniques and equipment known to experts in the field to give a homogeneous composition.

The quantity of lysozyme, preferably microencapsulated and gastroprotected, present in the composition which is the subject of the present invention, is comprised between 10 and 2,000 mg/day, preferably it is comprised between 400 and 1,000 mg/day, even more preferably between 500 and 800 mg/day, preferably in solid form; it is mixed with the probiotic bacteria, preferably in solid or lyophilised form, using techniques and equipment known to experts in the field to give a homogeneous composition. Lysozyme is available on the market in a pharmaceutically acceptable form.

### Experimental Section

The antimicrobial activity of 14 strains of lactobacilli, 4 strains of bifidobacteria and 2 strains of streptococci was tested against 4 biotypes of E coli. ATCC 8739, ATCC 10536, ATCC 35218 and ATCC 25922; these are available to the public. The values are expressed as inhibition halo (mm). The data are shown in Table 1.

To determine the antimicrobial activity, the strains were tested following the protocol set out in Santini et al. (2010) Characterization of probiotic strains: an application as feed additives in poultry against Campylobacter jejuni. Int J Food Microbiol. 2010 Jul 31;141 Suppl 1:S98-108. Epub 2010 Apr 8.

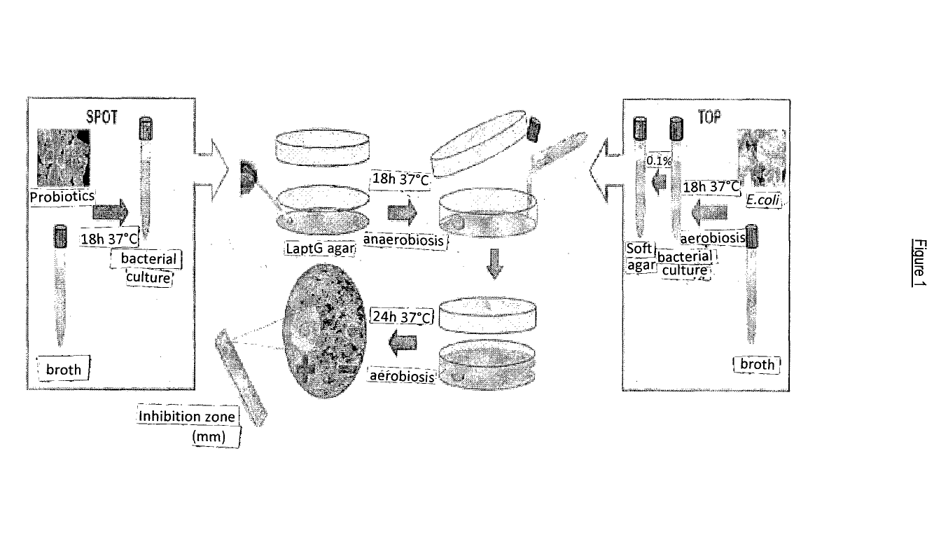

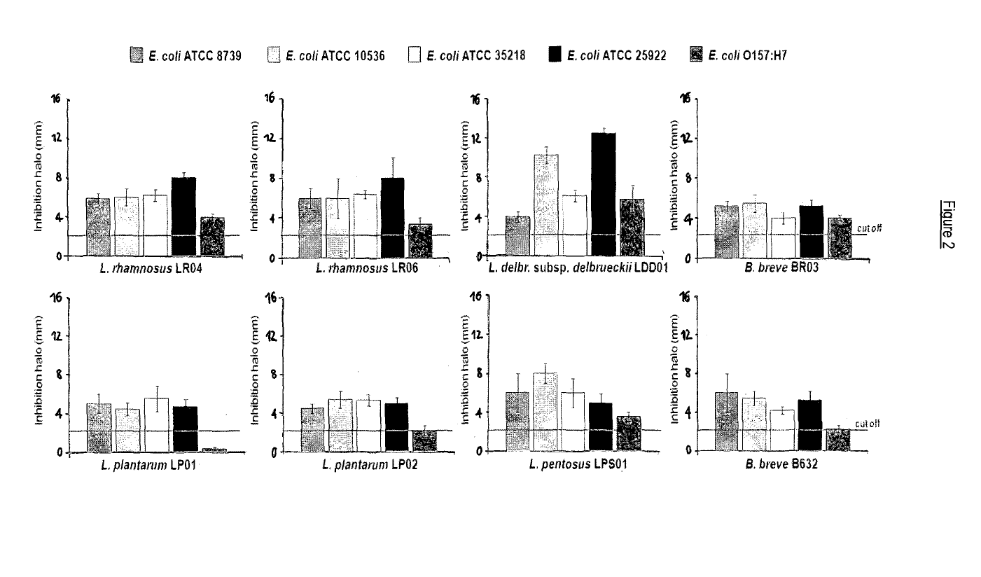

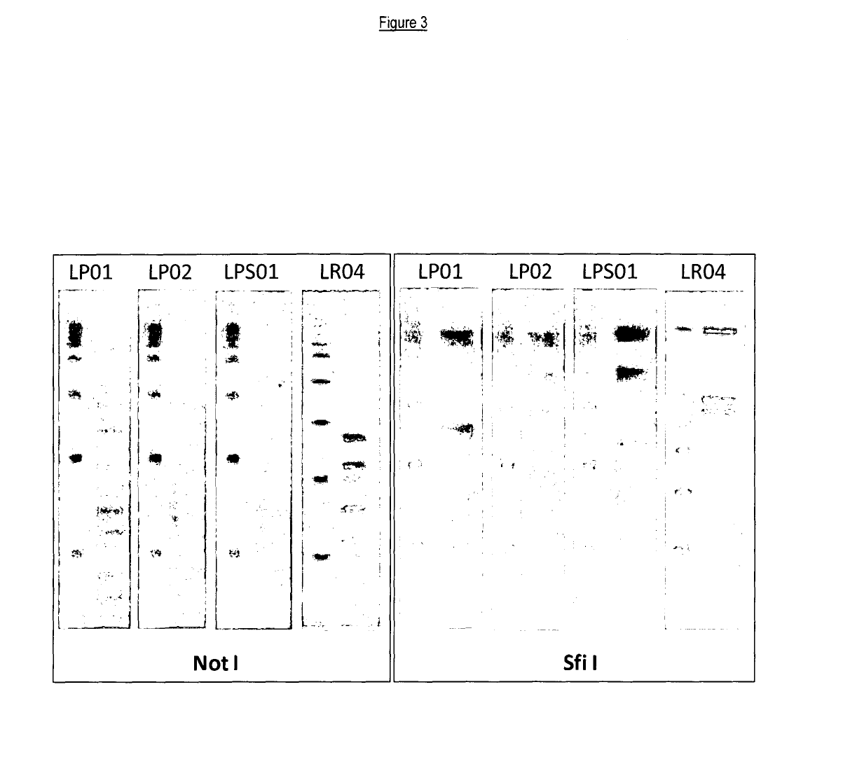

The strains of bacteria listed in Table 1 were cultivated in an MRS broth at 37° C. for 18 hours using a system for generating an anaerobic atmosphere (GasPack System). The different biotypes of E. coli were activated in Tryptic Soy Broth (TBS) and incubated aerobically at 37° C. for 18 hours. Before the experiments, the strains were subcultivated at least three times. For the spot agar test (a test known to experts in the field) the following procedure was carried out. In practice, 10 microlitres of a culture of the above-mentioned strains of lactobacilli grown at 37° C. overnight (culture broth+medium) were placed (spotted) on the surface of an agarised plate (containing the agarised TBS medium). The plate was incubated for 18 hours at 37° C. to allow the strain (spot) to develop. The biotypes of E. coli (0.1%) were incubated in Tryptic Soy soft agar and arranged on the spots in the plates referred to above. Once the top agar had solidified, the plates were inverted and incubated in conditions of aerobiosis at 37° C. for 24 hours. Each test was repeated twice. After 24 hours the plates were visually examined to observe and determine the inhibition zones. The presence of an inhibition zone of 2 mm or more around the spot of lactobacilli was considered a positive result (see FIG. 1 for a summary of the method used).

The same technique was used for the bifidobacteria as for the lactobacilli.

### Top Agar Technique:

E. coli strain streaked onto agarised Petri plate (McConkei Agar medium) incubated under aerobiosis for 24 h. 1 or 2 colonies are collected and dissolved in 1 ml of sterile water until they reach a turbidity value of 0.5 on the McFarland scale (known scale cited in reference tables).

100 μl of this suspension are inoculated into 50 ml of soft-agar medium (LAPTg with 0.7% agar) at a temperature of about 45° C. so as to keep it liquid.

3 ml of soft-agar are poured onto the surface of the plates previously prepared with the grown lactobacilli.

Dry and incubate at 37° C. under aerobiosis for 18-20 h.

Results read by measuring inhibition halo.

### Results

A. In vitro screening of the strains of the present invention against biotypes of E. coli.

The activity of inhibiting the growth of the E. coli was determined using the live bacterial cultures of the strains listed in Table 1. Positive results were obtained for all the strains listed in Table 2. In particular, the strains listed in Tables 3-5 were distinguished by their strong inhibition/reduction effect on the growth of all the biotypes of E. coli tested.

B. Selection of Strains of the Present Invention Having an Inhibitory Activity Against Toxinogenic E. coli O157:H7.

The strains listed in Table 1 were also tested against E. coli O157:H7. The inhibitory activity on the growth of E. coli O157:H7 was determined using the live bacterial cultures of the strains listed in Table 1.

As shown in FIG. 2, only 5 strains tested showed the capacity to inhibit/reduce the growth of the biotypes of E. coli tested, including the enterohaemorrhagic E. coli O157:H7 (CQ9485). In FIG. 2, the results are expressed as standard deviation (SD) of the inhibition halos, calculated on three different tests and allowing a cut-off of 2 mm.

From the experimental data reported above, it can be inferred that 6 lactobacilli and 2 bifidobacteria (Table 4) show a in vitro inhibition/reduction activity against the growth of four strains of E. coli (ATCC 8739 ATCC 10536 ATCC 35218 ATCC 25922), which are responsible for the production of a wide variety of biogenic amines such as for example mercaptan, histamine, cadaverine, putrescine and tyramine by decarboxylation of the amino acids. These biogenic amines are considered responsible for dangerous systemic intoxications. Furthermore, the presence of nitrites and nitrates can form N-nitrosamines which show strong mutagenic activity that can cause some types of cancer. Finally, the experimental data reported above show that five strains of bacteria tested are active against the pathogenic strain of E. coli O157:H7 (CQ9485) which is capable of producing one or more of the Shiga toxins that are very dangerous for human health and can even lead to death.

### Experimental Section

### Materials and Methods: Micro-Organisms and Cultural Conditions.

In the present study, 4 different species of probiotic bacteria were used, belonging to the genus Lactobacillus (Tables 6 and 7). All the bacteria were cultivated in Man, Rogosa and Sharpe (MRS) medium, (marketed by the company Becton Dickinson, Milan, Italy) at 37° C. for 18 hours, in conditions of anaerobiosis (GasPak, Becton Dickinson, Milan, Italy). The following 4 biotypes of E. coli were used as target strains: E. coli ATCC 8739, E. coli ATCC 10536, E. coli ATCC 35218 and E. coli ATCC 25922, purchased from the American Type Culture Collection (ATCC, Rockville, Md., USA). The enterohaemorrhagic strain E. coli O157:H7 (CQ 9485) was offered by the Gastroenterology Department, Ospedale Maggiore della Carità, Novara, Italy. Listeria monocytogenes ATCC 19112 was purchased from the American Type Culture Collection; Enterococcus sp. and Klebsiella sp. were isolated from samples of faeces of neonates affected by colic (University of Bologna, Italy). All the biotypes of E. coli, Enterococcus sp. and Klebsiella sp were cultivated in Tryptone Soy Broth (TSB) medium (Biotest, Rockaway, N.J., USA) and incubated aerobically at 37° C. for 18 hours. Listeria monocytogenes was cultivated in Fraser medium (Oxoid, Hampshire, United Kingdom) and incubated aerobically at 37° C. for 18 hours. All the bacterial strains were transplanted at least three times before being used in the inhibition experiments.

### Fermentative Profile Using API 50 CHL

The species of probiotic bacteria belonging to the genus Lactobacillus were cultivated for 18 hours at 37° C., washed twice in sterile water and centrifuged at 6000 rpm for 5 minutes. After resuspension in water, in a test tube containing 5 ml of water a quantity of sample was added such as to reach the opacity value 2 on the McFarland scale. A quantity of sample equal to double the quantity used previously was added to 10 ml of API 50 CHL medium (API systems, BioMerieux). 200 μl of the suspension were transferred to each well of the API 50 CH gallery. All the wells were recovered with paraffin oil to ensure anaerobiosis. The galleries were moistened and incubated at 37° C. Changes in the starting colour (purple) were checked after 1 and 2 days of incubation.

### Primers and Species-specific PCR Assay

The genomic DNA of each strain of Lactobacillus is extracted with the MicroLysis™ kit (Labogen, Rho, Italy). Specifically, 2 μl of each bacterial culture is added to the lysis buffer and lysis is performed with the Gene Amp PCR System 9700 thermal cycler (Perkin-Elmer, Monza, Italy) following the manufacturer's instructions.

For the strains of L. plantarum to L. pentosus, the primers used for the PCR reaction were designed on the recA gene, while for the strain of L. rhamnosus, the intergenic region comprised between 16S and 23S of the ribosomal RNA (Table 6) was used. All the primers were synthesised by the firm Sigma-Aldrich.

For the amplification, a mix (25 μl) was made up with 2× GoTaq Green Master Mix (Promega), 1 μl of lysed DNA and the set of species-specific primers (0.30 mM). The PCR cycles were performed with the Gene Amp PCR System 9700 thermal cycler. The amplification programs are shown in Table 6.

### PFGE

The strains of Lactobacillus are cultivated in MRS medium until an optical density at 600 nm (OD₆₀₀) is reached of 0.8. After washing in the buffer solution of 10 mM Tris, 20 mM NaCl, 50 mM EDTA, pH 7.2, the cells are resuspended in 300 μl of the same buffer solution. After the addition of 300 μl of agarose 2% (Bio-Rad), the samples are poured into the moulds.

The plugs are incubated for 18 hours at 37° C. in the lysis buffer solution (6 mM Tris, 1 M NaCl, 100 mM EDTA, 1% sarcosyl, 0.2% deoxycholate, pH 7.6), with the addition of 2.5 mg/ml of lysozyme (Sigma) and 4 U/ml of mutanolysin (Sigma). Treatment with Proteinase K (1 mg/ml) was performed in buffer solution 100 mM EDTA, 1% sarcosyl, 0.2% deoxycholate at pH 8.0 for 24 h at 50° C. The plugs are washed 4 times with the buffer solution 20 mM Tris, 50 mM EDTA at pH 8.0, adding in the first 2 washes 1 mM phenylmethylsulfonyl fluoride (Sigma). Before digestion with the restriction enzyme, the plugs are washed twice with the TE buffer and then equilibrated for 1 hour in the appropriate restriction enzyme buffer. The digestions with the restriction enzymes are carried out for 18 hours at 37° C. for Notl and at 50° C. with Sfil. The electrophoretic cycle is performed with the CHEF DR III apparatus (Bio-Rad) using a 1% agarose gel (Bio-Rad) in a 0.5 M TBE buffer.

The cycle is set with the following parameters: Notl (switch time: 1-11 s, voltage 6 V/cm, buffer temperature 14° C., cycle time 27 h); Sfil (switch time: 1-15 s, voltage 5 V/cm, buffer temperature 14° C., cycle time 22 h). The agarose gel is stained with ethidium bromide (0.5 mg/ml) and viewed by means of UV light.

### Assessment of In Vitro Inhibitory Activity

The antimicrobial activity against E. coli was assessed using the protocol described by Santini C. et al. ²⁶. Briefly, 10 μl of each LAB culture with optical density at 600 nm of about 1.0 are poured (drop) onto the surface of a Petri plate agarised with LAPTg medium and incubated anaerobically for 24 hours at 37° C. to allow bacterial growth (spot). The target strain of E. coli (0.1%) is inoculated in Tryptic Soy soft agar medium and poured onto the plates containing the spots. After solidification, the plates are inverted and incubated under conditions of aerobiosis for 24 hours at 37° C.

Each experiment was performed in duplicate. The presence of a halo free of growth of the pathogen under examination (inhibition halo) was considered a positive result.

The quantification of the inhibitory activity of the probiotic in relation to different strains of E. coli was performed in the laboratories of the Research and Development division of the company Biolab Research, MofinAlce Group, while the quantification relating to the enterohaemorrhagic strain E. coli O157:H7 was carried out by the specialist staff of the private clinic “I Cedri”, Fara Novarese, Italy.

Antimicrobial activity against Listeria monocytogenes, Enterococcus sp. and Klebsiella sp. was assessed using the disc diffusion method. Briefly, the LABs were cultivated in MRS medium for 24 hours at 37° C., then washed and resuspended in fresh medium. The pathogen is uniformly seeded onto the surface of Petri plate containing the agarised medium specific for the pathogenic species under examination. Different paper discs are positioned on the surface of the plate with the pathogen and 100 μl of LAB are then absorbed onto each disc. The plates are incubated at 37° C. for 24 hours. The presence of a halo free of growth of the pathogen under examination (inhibition halo) was considered a positive result. The results are expressed as inhibition halos (mm). The threshold for determining the significance of the result is 2 mm.

### Results

### In Vitro Inhibitory Activity of Probiotic Strains Against Pathogenic Bacteria

The antimicrobial activity of some strains of lactobacilli against pathogenic bacteria is multi-factorial and includes the production of hydrogen peroxide, lactic acid, known molecules such as bacteriocins and unknown molecules stable to heat.

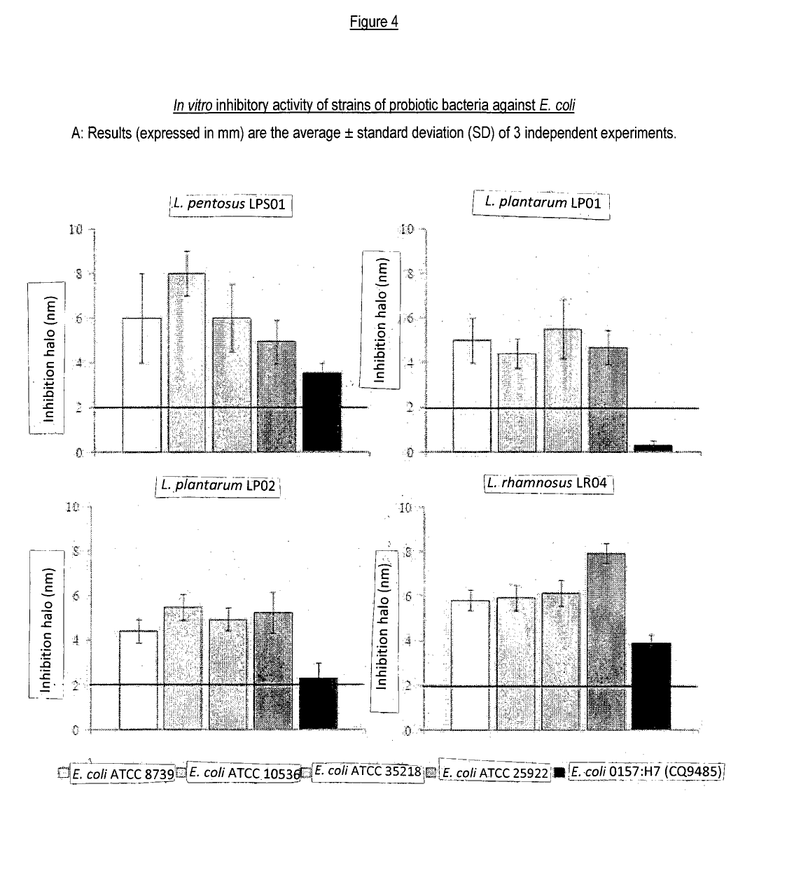

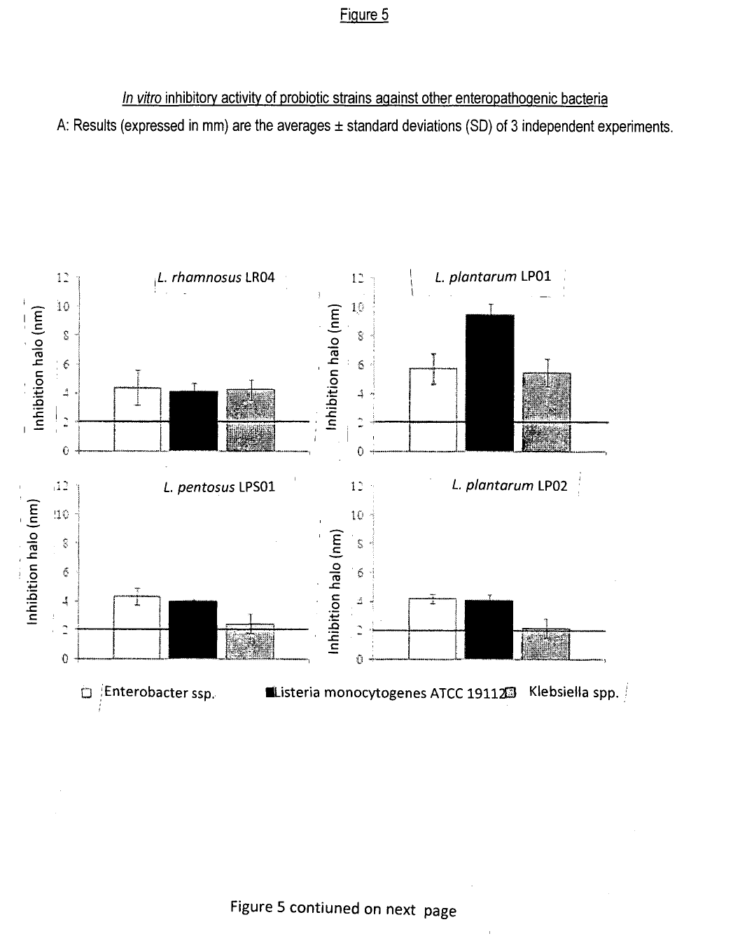

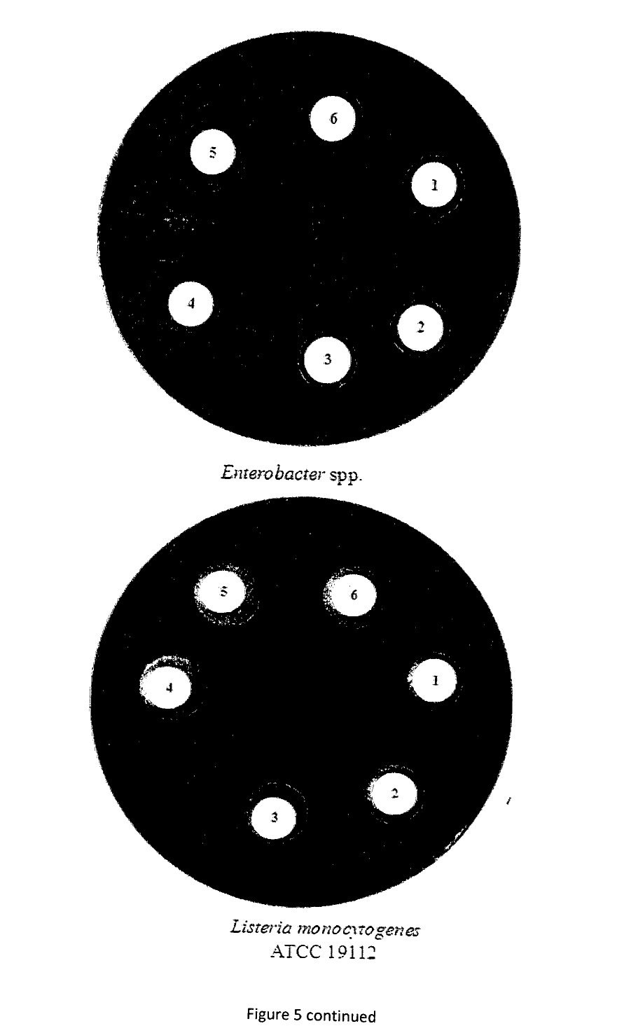

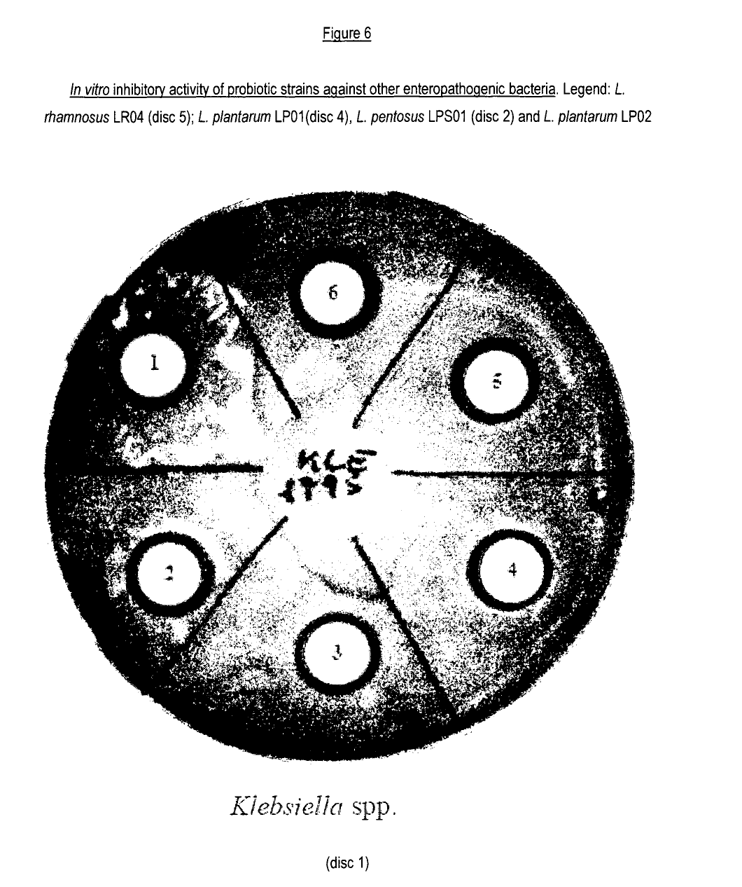

As shown in FIG. 4, the results obtained by the spot method on agar and live cells highlighted the fact that all the bacteria tested show inhibitory activity in relation to the four strains of E. coli. The probiotic strains which display the greatest inhibitory activity are L. rhamnosus LR04 and L. pentosus LPS01. The data obtained using as target the enterohaemorrhagic strain Escherichia coli serotype O157:H7, too, showed the best inhibitory activity in the presence of L. rhamnosus LR04 and L. pentosus LPS01 (FIG. 4).

To better characterise the probiotic strains under examination, the analysis was widened to other pathogenic species which strike the human gastro-intestinal tract, using the disc diffusion method. The strain L. plantarum LP01 unexpectedly showed the best inhibitory capacity against all the enteropathogenic bacteria tested, in particular against Listeria monocytogenes.

TABLE 6

| Bacterial | | | | |
|---|---|---|---|---|
| strain | name | sequence (5′-3′) | cycle | reference |
| L. rhamnosus | RHA | GCGATGCGAATTTCTATTATT | 5′95° C., (30″ 94° C., | Appl. And Environ. |
| | PRI | CAGACTGAAAGTCTGACGGT | 30″ 58° C., 30″ | Microb. 66 (2000) |
| | | | 72° C.) × 30, 7′ 72° C. | 297-303 |
| L. plantarum | PLAN-f | CCGTTTATGCGGAACACCTA | 3′94° C., (30″ 94° C., | Appl. And Environ. |
| | P-REV | TCGGGATTACCAAACATCAC | 10″ 56° C., 30″ | Microb. 67 (2001) |
| | | | 72° C.) × 30, 7′ 72° C. | 3450-3454 |
| L. pentosus | PENT-f | CAGTGGCGCGGTTGATATC | 3′94° C., (30″ 94° C., | Appl. And Environ. |
| | P-REV | TCGGGATTACCAAACATCAC | 10″ 56° C., 30″ | Microb. 67 (2001) |
| | | | 72° C.) × 30, 7′ 72° C. | 3450-3454 |

TABLE 7

| | Code/Filing no. |
|---|---|
| Strain | depositio |
| L. pentosus | LPS01 |
| L. plantarum | LP01 |
| L. plantarum | LP02 |
| L. rhamnosus | LR04 |

1. A method of preventive and/or curative treatment of infections and/or pathologies in an individual, the method comprising
administering to the individual a pharmaceutical or dietary composition or a supplement or a medical device comprising at least one bacterial strain belonging to one or more species selected from the group consisting of Lactobacillus delbrueckii, Lactobacillus de/brueckii subsp. de/brueckii, Lactobacillus plantarum, Lactobacillus rhamnosus, Lactobacillus pentosus, Lactobacillus reuteri and Bifidobacterium breve which is capable of producing bacteriocins and/or metabolites and/or oxygenated water, the at least one bacterial strain comprised in an amount effective for preventive and/or curative treatment of infections and/or pathologies connected with pathogens belonging to the species E. coli, Clostridium difficile, Listeria monocytogenes, Enterococcus sp. and Klebsiella sp.

2. The method according to claim 1, wherein the infections and/or pathologies connected with pathogens are infections and/or pathologies connected with E. coli.

3. The method according to claim 1, wherein the the infections and/or pathologies connected with pathogens are infections and/or pathologies connected with Listeria monocytogenens strain ATCC 19112.

4. The method according to claim 1, wherein the at least one bacterial strain is selected from the group consisting of:
B. breve 8R03 DSM 16604
B. breve 8632 DSM 24706
L. rhamnosus LR04 DSM 16605
L. rhamnosus LR06 DSM 21981
L. plantarum LP01 LMG P-21021
L. plantarum LP02 LMG P-21 020
L. plantarum LP03 LMG P-21 022
L. plantarum LP04 LMG P-21 023
L. pentosus LPS01 DSM 21980
L. de/br. subsp. delbrueckii LDDO 1 DSM 221 06
L. reuteri LRE01 DSM 23877
L. reuteri LRE03 DSM 23879.

5. The method according to claim 4, wherein the at least one bacterial strain is selected from the group consisting of:
B. breve 8R03 DSM 16604
B. breve 8632 DSM 24 706
L. rhamnosus LR04 DSM 16605
L. rhamnosus LR06 DSM 21981
L. p/antarum LP01 LMG P-21 021
L. plantarum LP02 LMG P-21 020
L. pentosus LPS01 DSM 21980
L. delbr. subsp. delbrueckii LDD01 DSM 22106.

6. The method according to claim 5, wherein the at least one bacterial strain is selected from the group consisting of strains L. rhamnosus LR04 DSM 16605 and L. pentosus LPS01 DSM 21980.

7. The method according to claim 5, wherein the at least one bacterial strain is is L. plantarum LP01 LMG P-21021.

8. The method according to claim 1, wherein said pharmaceutical or dietary composition or a supplement or a medical device further comprises acetylcysteine.

9. The method according to claim 1, wherein said pharmaceutical or dietary composition or a supplement or a medical device further comprises lysozyme.

10. The method according to claim 1, wherein said pharmaceutical or dietary composition or a supplement or a medical device further comprises lysozyme microencapsulated in a lipid matrix having a melting point comprised from 30° C. to 80° C.

11. The method according to claim 1, wherein said pharmaceutical or dietary composition or a supplement or a medical device further comprises acetylcysteine and lysozyme microencapsulated in a lipid matrix and wherein said composition has a melting point from 30° C. to 80° C.

12. The method according to claim 5, wherein said pharmaceutical or dietary composition or a supplement or a medical device further comprises at least one prebiotic fibre and/or carbohydrates with bifidogenic action.

13. The method according to claim 1, wherein the infections and/or pathologies connected with pathogens, are infections and/or pathologies connected with a strain of bacteria selected from the group consisting of: E. coli serotype 0157:H7 or E. coli 0104:H4, and strains E. coli ATCC 8739, E. coli ATCC 10536, E. coli ATCC 35218 and E. coli ATCC 25922.

14. The method according to claim 4, wherein said composition administered consists of two or three or four or five or six strains belonging to one or more species.

15. The method according to claim 6, wherein the infections and/or pathologies connected with pathogens, are infections and/or pathologies connected with a strain of bacteria selected from the group consisting of: c4 E. coli serotype 0157:H7 or E. coli 0104:H4.

16. The method according to claim 7, wherein the infections and/or pathologies connected with pathogens, are infections and/or pathologies connected with pathogens belonging to the species Listeria monocytogenes.

17. The method according to claim 10, wherein the lipid matrix is of natural origin.

18. The method according to claim 10, wherein the pharmaceutical or dietary composition or a supplement or a medical device further comprises lysozyme microencapsulated in a lipid matrix having a melting point comprised from 50° C. to 60° C.

19. The method according to claim 11, wherein the lipid matrix is of natural origin.

20. The method according to claim 11, wherein said pharmaceutical or dietary composition or a supplement or a medical device further comprises acetylcysteine and lysozyme microencapsulated in a lipid matrix having a melting point comprised from 50° C. to 60° C.


SEQUENCE LISTING (6 sequences)

SEQ ID NO: 1 - DNA, 21 bp, artificial sequence
  Synthetic polynucleotide
gcgatgcgaatttctattatt

SEQ ID NO: 2 - DNA, 20 bp, artificial sequence
  Synthetic polynucleotide
cagactgaaagtctgacggt

SEQ ID NO: 3 - DNA, 20 bp, artificial sequence
  Synthetic polynucleotide
ccgtttatgcggaacaccta

SEQ ID NO: 4 - DNA, 20 bp, artificial sequence
  Synthetic polynucleotide
tcgggattaccaaacatcac

SEQ ID NO: 5 - DNA, 19 bp, artificial sequence
  Synthetic polynucleotide
cagtggcgcggttgatatc

SEQ ID NO: 6 - DNA, 20 bp, artificial sequence
  Synthetic polynucleotide
tcgggattaccaaacatcac